(19) United States
(12) Patent Application Publication
(10) Pub. No.: US 2023/0181848 A1
(43) Pub. Date: Jun. 15, 2023

(54) METHOD AND DEVICE FOR MANUFACTURING AN INHALER ARTICLE

(71) Applicant: PHILIP MORRIS PRODUCTS S.A., Neuchatel (CH)

(72) Inventor: Fiorenzo Draghetti, Medicina (IT)

(21) Appl. No.: 17/924,724

(22) PCT Filed: May 18, 2021
(86) PCT No.: PCT/EP2021/063064

§ 371 Date: Nov. 11, 2022

(30) Foreign Application Priority Data
May 18, 2020 (EP) 20175216.9

(51) Int. Cl. A61M 15/06; A61M 15/00

(52) U.S. Cl. CPC A61M 15/06; A61M 15/003; A61M 2202/064

(57) ABSTRACT

The invention relates to a method for manufacturing an inhaler article, the inhaler article comprising a body, a capsule cavity holding a capsule, a mouthpiece element and a deformable tubular element having an open distal end, the method comprising pre-treating the distal end of the deformable tubular element to obtain a pre-treated portion with reduced structural stability, and folding the pre-treated portion inwards by at least 90 degrees to at least partially close the distal end. The invention relates also to a device for manufacturing an inhaler article and to an inhaler article obtainable by the device.

The present invention relates to a method and a device for manufacturing an inhaler article, wherein the inhaler article comprises a body, a capsule cavity holding a capsule, a mouthpiece element and a deformable tubular element having an open end. The method comprises pre-treating and folding the distal end of the deformable tubular element inwards by at least 90 degrees to at least partially close the distal end of the deformable tubular element.

Dry powder inhalers are not always fully suitable to provide dry powder particles to the lungs at inhalation or air flow rates that are within conventional smoking regime inhalation or airflow rates. Dry powder inhalers may be complex to operate or may involve moving parts. Dry powder inhalers often strive to provide an entire dry powder dose or capsule load in a single breath.

It would be desirable to provide a method and a device for reproducibly and automatically manufacturing an inhaler article.

It would be desirable to provide a method and a device for manufacturing an inhaler article at sufficiently high speed.

It would be desirable to provide a method and a device for manufacturing an inhaler article, wherein the manufacturing method can be implemented in existing manufacturing lines used for production of aerosol-generating articles.

According to an embodiment of the present invention there is provided a method for manufacturing an inhaler article, wherein the inhaler article comprises a body, a capsule cavity holding a capsule, a mouthpiece element and a deformable tubular element having an open end. The method comprises the steps of pre-treating the distal end of the deformable tubular element to obtain a pre-treated portion with reduced structural stability, and folding the pre-treated portion inwards by at least 90 degrees to at least partially close the distal end.

The present invention provides a simple and efficient method for manufacturing an inhaler article comprising a deformable tubular element that defines a capsule cavity and that is folded to have an at least partially closed distal end.

The present invention allows using standard foldable materials leading to a cost efficient production of inhaler articles.

Moreover, the method of the present invention is fast and has a high reproducibility. The method can therefore be used in industrial and automatic manufacturing of inhaler articles. Moreover, the present method can be implemented in existing manufacturing lines used for production of aerosol-generating articles.

The term “deformable” should be understood to mean that the shape of the deformable element is changeable. The deformation of the deformable element may include elastic deformation, where the deformable element reverts back to the closed configuration in the absence of a force being applied to it. Alternately, the deformation of the deformable element may include plastic deformation where the deformable element is held in the open configuration after the application of a force.

At least a portion of the deformable element may be formed of a foldable material. The deformable element may comprise a fan fold. At least a portion of the deformable element may be formed of cellulosic material. At least a portion of the deformable element may be formed of paper.

Advantageously, forming the deformable element of a foldable material allows the deformable element to be breached or opened reliably. A foldable material may also improve the assembly of the capsule cavity and provide for high speed assembly of the inhaler article.

Advantageously, the deformable element formed of cellulose material or paper is substantially biodegradable and may reduce the environmental impact of the inhaler article.

The deformable element may define at least a portion of a longitudinal sidewall of the capsule cavity. The deformable element may define a majority of the capsule cavity. The deformable element may define the upstream boundary and the sidewalls of the capsule cavity.

Advantageously, the deformable element may provide a protective cover or hygiene barrier for the retained capsule and inhaler article prior to consumption of the inhaler article.

A wrapping layer may circumscribe the mouthpiece element and the deformable element. A wrapping layer may join the mouthpiece element, capsule cavity, and the deformable element in serial axial abutment. The deformable element may extend beyond the wrapping layer. The deformable element may extend beyond the wrapping layer in a range from about 0.5 millimetres to about 5 millimetres, or from about 1 millimetre to about 4 millimetres, or about 2 millimetres to about 3 millimetres. The wrapping layer may be formed of a cellulose material or paper.

Advantageously, a wrapping layer formed of cellulose material is substantially biodegradable and may reduce the environmental impact of the inhaler article. Joining inhaler article elements with a wrapping layer provides for high speed assembly of the inhaler article.

The capsule cavity and deformable element have substantially equal inner diameters in a range from about 6 millimetres to about 8 millimetres.

The capsule may contain pharmaceutically active particles. For instance, the pharmaceutically active particles may comprise nicotine. The pharmaceutically active particles may have a mass median aerodynamic diameter of about 5 micrometres or less, or in a range from about 0.5 micrometres to about 4 micrometres, or in a range from about 1 micrometres to about 3 micrometres.

The terms “proximal” and “distal” are used to describe the relative positions of components, or portions of components of the inhaler article or system. Inhaler articles, according to the invention have a proximal end. In use, the nicotine particles exit the proximal end of the inhaler article for delivery to a user. The inhaler article has a distal end opposing the proximal end. The proximal end of the inhaler article may also be referred to as the mouth end.

The inhaler article may resemble a smoking article or cigarette in size and shape. The inhaler article may have an elongated body extending along the longitudinal axis of the inhaler article. The inhaler body may have a substantially uniform outer diameter along the length of the elongated body. The inhaler article may have a circular cross-section that may be uniform along the length of the elongated body. The inhaler body may have an outer diameter in a range from about 6 millimetres to about 10 millimetres, or from about 7 millimetres to about 10 millimetres, or about 7 millimetres to about 9 millimetres, or about 7 millimetres to about 8 millimetres or about 7.3 millimetres. The inhaler article may have a length (along the longitudinal axis) in a range from about 40 millimetres to about 80 millimetres, or from about 40 millimetres to about 70 millimetres, or about 40 millimetres to about 50 millimetres, or about 48 millimetres.

The mouthpiece element located downstream of the capsule cavity may extend from the capsule cavity to the mouthpiece end of the inhaler article. The mouthpiece element may have a length in a range from about 10 millimetres to about 30 millimetres, preferably from about 15 millimetres to about 25 millimetres and more preferably from about 20 millimetres to about 22 millimetres. The mouthpiece element may have a diameter in a range from about 6 millimetres to about 10 millimetres, or from about 7 millimetres to about 10 millimetres, or about 7 millimetres to about 9 millimetres, or about 7 millimetres to about 8 millimetres or about 7.1 millimetres.

The mouthpiece element may have a filtering function. The mouthpiece element may comprise a filter element. The filter element may extend substantially over the full length of the mouthpiece element.

The deformable element is configured to deform and expose the capsule cavity. The deformable element is configured to be breached or opened to expose the capsule cavity. The deformable element is configured to expose substantially the entire open diameter of the capsule cavity. The deformable element is configured to expose the entire open diameter of the capsule cavity.

The deformable element may define at least a portion of a longitudinal sidewall of the capsule cavity. The deformable element may define a majority of the capsule cavity. The deformable element may define a closed distal end or upstream end of the capsule cavity.

The deformable element may be formed of cellulosic material. At least a portion of the deformable element may be formed of paper. The deformable element may provide a barrier to reduce or prevent contaminants or foreign material from entering the capsule cavity.

The capsule cavity sidewall extends parallel with the longitudinal axis of the inhaler article. The deformable element may define a closed distal end or upstream end of the capsule cavity and at least a portion of the capsule cavity sidewall.

The deformable element may define a tubular element having a closed upstream end. The deformable element may define a closed distal end or upstream end of the capsule cavity and at least 50 percent of the capsule cavity sidewall. The deformable element may define a closed distal end or upstream end of the capsule cavity and at least 75 percent of the capsule cavity sidewall. The deformable element may define a closed distal end or upstream end of the capsule cavity and the entire capsule cavity sidewall. The deformable element may define the entire capsule cavity except for the downstream boundary surface defined by the mouthpiece element. The deformable element may be a paper layer extending from the mouthpiece element to the closed upstream end.

Inhalation air flows through the center of the deformable element directly into the capsule cavity once the deformable element is breached or opened. The deformable element may have a diameter that is substantially equal to the inner diameter of the capsule cavity.

The deformable element may have an outer diameter in a range from about 6 millimetres to about 8 millimetres or from about 7.0 millimetres to about 7.1 millimetres. The deformable element may have an inner diameter in a range from about 6 millimetres to about 7.2 millimetres or from about 6.5 millimetres to about 6.7 millimetres.

The deformable element may be formed of paper. The deformable element may be formed of one or more paper layers. The deformable element may be formed of paper having a weight in a range of about 50 grams per square meter to about 150 grams per square meter, or from about 75 grams per square meter to about 125 grams per square meter, or from about 90 grams per square meter to about 110 grams per square meter.

The deformable element may have a thickness in a range from about 50 micrometres about 200 micrometres, or from about 100 micrometres to about 150 micrometres, or from about 110 micrometres to about 130 micrometres.

Once breached or opened, the deformable element may define an opening having an open diameter that is at least about 80 percent or at least about 90 percent of the diameter of the capsule cavity.

The deformable element may be easily breached to allow inhalation air to enter the capsule cavity. For instance, the deformable element may be configured to breach upon manual insertion of the inhaler article into a holder by a user without the use of additional tools for assisting the application of force by a user. The deformable element may breach or open to expose substantially the entire upstream end of the capsule cavity. The deformable element may provide a protective cover or hygiene barrier for the retained capsule and inhaler article prior to consumption of the inhaler article.

A wrapping layer may define the body of the inhaler article. The wrapping layer may circumscribe the mouthpiece element and the deformable element. The wrapping layer may join the mouthpiece element and the deformable element. The wrapping layer may join the mouthpiece element, and deformable element in serial axial abutment. The wrapping layer may be formed of a cellulose material.

The deformable element may extend beyond the wrapping layer. The deformable element may extend beyond the wrapping layer in a range from about 0.5 millimetres to about 5 millimetres, or from about 1 millimetre to about 4 millimetres, or about 2 millimetres to about 3 millimetres.

The capsule cavity may define a cylindrical space configured to contain a capsule. For example, the capsule may have an obround shape or a circular cross-section. The capsule cavity may have a substantially uniform or uniform diameter along the length of the capsule cavity. The capsule cavity may have a fixed cavity length. The capsule cavity has a cavity inner diameter, orthogonal to the longitudinal axis, and the capsule has a capsule outer diameter. The capsule cavity may be sized to contain an obround capsule. The capsule cavity may have a substantially cylindrical or cylindrical cross-section along the length of the capsule cavity. The capsule cavity may have a uniform inner diameter. The capsule may have an outer diameter that is about 80 percent to about 95 percent of the inner diameter of the capsule cavity. The configuration of the capsule cavity relative to the capsule may promote limited movement of the capsule during activation or piercing of the capsule.

The capsule cavity may be defined by the deformable element having a diameter in a range from about 6 millimetres to about 8 millimetres mm or about 6.6 millimetres.

The capsule may contain pharmaceutically active particles. For instance, the pharmaceutically active particles may comprise nicotine. The pharmaceutically active particles may have a mass median aerodynamic diameter of about 5 micrometres or less, or in a range from about 0.5 micrometres to about 4 micrometres, or in a range from about 1 micrometres to about 3 micrometres.

The capsule may contain nicotine particles comprising nicotine (also referred to as “nicotine powder” or “nicotine particles”) and optionally particles comprising flavour (also referred to as “flavour particles). The capsule may contain a predetermined amount of nicotine particles and optional flavour particles. The capsule may contain enough nicotine particles to provide at least 2 inhalations or “puffs”, or at least about 5 inhalations or “puffs”, or at least about 10 inhalations or “puffs”. The capsule may contain enough nicotine particles to provide from about 5 to about 50 inhalations or “puffs”, or from about 10 to about 30 inhalations or “puffs”. Each inhalation or “puff” may deliver from about 0.1 mg to about 3 mg of nicotine particles to the lungs of the user or from about 0.2 milligrams to about 2 milligrams of nicotine particles to the lungs of the user or about 1 milligram of nicotine particles to the lungs of the user.

The nicotine particles may have any useful concentration of nicotine based on the particular formulation employed. The nicotine particles may have at least about 1 weight-percent nicotine up to about 30 weight-percent nicotine, or from about 2 weight-percent to about 25 weight-percent nicotine, or from about 3 weight-percent to about 20 weight-percent nicotine, or from about 4 weight-percent to about 15 weight-percent nicotine, or from about 5 weight-percent to about 13 weight-percent nicotine. Preferably, about 50 to about 150 micrograms of nicotine may be delivered to the lungs of the user with each inhalation or “puff”.

The capsule may hold or contain at least about 5 milligrams of nicotine particles or at least about 10 milligrams of nicotine particles. The capsule may hold or contain less than about 900 milligrams of nicotine particles, or less than about 300 milligrams of nicotine particles, or less than 150 milligrams of nicotine particles.

The capsule may hold or contain from about 5 milligrams to about 300 milligrams of nicotine particles or from about 10 milligrams to about 200 milligrams of nicotine particles.

When flavour particles are blended or combined with the nicotine particles within the capsule, the flavour particles may be present in an amount that provides the desired flavour to each inhalation or “puff” delivered to the user.

The nicotine particles may have any useful size distribution for inhalation delivery preferentially into the lungs of a user. The capsule may include particles other than the nicotine particles. The nicotine particles and the other particles may form a powder system.

The capsule may hold or contain at least about 5 milligrams of a dry powder (also referred to as a powder system) or at least about 10 milligrams of a dry powder. The capsule may hold or contain less than about 900 milligrams of a dry powder, or less than about 300 milligrams of a dry powder, or less than about 150 milligrams of a dry powder. The capsule may hold or contain from about 5 milligrams to about 300 milligrams of a dry powder, or from about 10 milligrams to about 200 milligrams of a dry powder, or from about 25 milligrams to about 100 milligrams of a dry powder.

The dry powder or powder system may have at least about 40 percent, or at least about 60 percent, or at least about 80 percent, by weight of the powder system comprised in nicotine particles having a particle size of about 5 micrometres or less, or in a range from about 1 micrometre to about 5 micrometres.

The particles comprising nicotine may have a mass median 5 aerodynamic diameter of about 5 micrometres or less, or in a range from about 0.5 micrometres to about 4 micrometres, or in a range from about 1 micrometre to about 3 micrometres or in a range from about 1.5 micrometres to about 2.5 micrometres. The mass median aerodynamic diameter is preferably measured with a cascade impactor.

The particles comprising flavour may have a mass median aerodynamic diameter of about 20 micrometres or greater, or about 50 micrometres or greater, or in a range from about 50 to about 200 micrometres, or from about 50 to about 150 micrometres. The mass median aerodynamic diameter is preferably measured with a cascade impactor.

The dry powder may have a mean diameter of about 60 micrometres or less, or in a range from about 1 micrometre to about 40 micrometres, or in a range from about 1.5 micrometres to about 25 micrometres. The mean diameter refers to the mean diameter per mass and is preferably measured by laser diffraction, laser diffusion or an electronic microscope.

Nicotine in the powder system or nicotine particles may be a pharmaceutically acceptable free-base nicotine, or nicotine salt, or nicotine salt hydrate. Useful nicotine salts or nicotine salt hydrates include nicotine pyruvate, nicotine citrate, nicotine aspartate, nicotine lactate, nicotine bitartrate, nicotine salicylate, nicotine fumarate, nicotine mono-pyruvate, nicotine glutamate or nicotine hydrochloride, for example. The compound combining with nicotine to form the salt or salt hydrate may be chosen based on its expected pharmacological effect.

The nicotine particles preferably include an amino acid. Preferably, the amino acid may be leucine such as L-leucine. Providing an amino acid such as L-leucine with the particles comprising nicotine, may reduce adhesion forces of the particles comprising nicotine and may reduce attraction between nicotine particles and thus reduce agglomeration of nicotine particles.

Similarly, adhesion forces to particles comprising flavour may also be reduced thus agglomeration of nicotine particles with flavour particles is also reduced. The powder system described herein thus may be a free-flowing material and possess a stable relative particle size of each powder component even when the nicotine particles and the flavour particles are combined.

Preferably, the nicotine may be a surface modified nicotine salt where the nicotine salt particle comprises a coated or composite particle. A preferred coating or composite material may be L-leucine. One particularly useful nicotine particle may be nicotine bi 5 tartrate with L-leucine.

The powder system may include a population of flavour particles. The flavour particles may have any useful size distribution for inhalation delivery selectively into the mouth or buccal cavity of a user.

The powder system may have at least about 40 percent, or at least about 60 percent, or at least about 80 percent, by weight of the population of flavour particles of the powder system comprised in particles having a particle size of about 20 micrometres or greater. The powder system may have at least about 40 percent or at least about 60 percent, or at least about 80 percent, by weight of the population of flavour particles of the powder system comprised in particles having a particle size of about 50 micrometres or greater. The powder system may have at least about 40 percent or at least about 60 percent, or at least about 80 percent, by weight of the population of flavour particles of the powder system comprised in particles having a particle size in a range from about 50 micrometre to about 150 micrometres.

The particles comprising flavour may include a compound to reduce adhesion forces or surface energy and resulting agglomeration. The flavour particle may be surface modified with an adhesion reducing compound to form a coated flavour particle. One preferred adhesion reducing compound may be magnesium stearate. Providing an adhesion reducing compound such as magnesium stearate with the flavour particle, especially coating the flavour particle, may reduce adhesion forces of the particles comprising flavour and may reduce attraction between flavour particles and thus reduce agglomeration of flavour particles. Thus, agglomeration of flavour particles with nicotine particles may also be reduced. The powder system described herein thus may possess a stable relative particle size of the particles comprising nicotine and the particles comprising flavour even when the nicotine particles and the flavour particles are combined. The powder system preferably may be free flowing.

Conventional formulations for dry powder inhalation contain carrier particles that serve to increase the fluidization of the active particles since the active particles may be too small to be influenced by simple airflow though the inhaler. The powder system may comprise carrier particles. These carrier particles may be a saccharide such as lactose or mannitol that may have a particle size greater than about 50 micrometres. The carrier particles may be utilized to improve dose uniformity by acting as a diluent or bulking agent in a formulation.

The powder system utilized with the nicotine powder delivery system described herein may be carrier-free or substantially free of a saccharide such as lactose or mannitol. Being carrier-free or substantially free of a saccharide such as lactose or mannitol may allow the nicotine to be inhaled and delivered to the user's lungs at inhalation or airflow rates that are similar to typical smoking regime inhalation or airflow rates.

The nicotine particles and a flavour may be combined in a single capsule. As described above, the nicotine particles and a flavour may each have reduced adhesion forces that result in a stable particle formulation where the particle size of each component does not substantially change when combined. Alternatively, the powder system includes nicotine particles contained within a single capsule and the flavour particles contained within a second capsule.

The nicotine particles and flavour particles may be combined in any useful relative amount so that the flavour particles are detected by the user when consumed with the nicotine particles.

Preferably, the nicotine particles and flavour particles form at least about 90 weight-percent or at least about 95 weight-percent or at least about 99 weight-percent or 100 weight-percent of the total weight of the powder system.

The pre-treating step of the method of the present invention may comprise crimping the edge of the distal end of the deformable tubular element. Upon crimping the edge of the deformable tubular element is folded along one or more lines running essentially parallel to the axial direction of the inhaler article.

The pre-treating step of the method of the present invention may comprise cutting the edge of the distal end of the deformable tubular element along one or more lines running generally parallel to the axial direction of the inhaler article.

The pre-treating step of the method of the present invention may comprise scoring the edge of the distal end of the deformable tubular element along one or more lines running generally parallel to the axial direction of the inhaler article. Upon scoring the deformable element is provided with a discontinuous cutting line.

The length of the crimping, scoring or cutting lines may be in a range from 0.5 to 5 millimeter, preferably from about 1 to 4 millimeters, and preferably from about 2.5 to 3.5 millimeters. Generally the length of these lines determines the length of the pre-treated portion with reduced structural stability.

The required length of the pre-treated portion depends on the diameter of the inhaler article.

Typical inhaler articles may have a diameter of 7.2 millimeters. For such articles the useful length of the pretreated portion may be at least about 3 millimeters and may be at most equal to the radius (3.6 millimeters). With a pre-treated portion of such dimensions, a sufficient closure of the distal end of the deformable tubular element may be achieved.

During the pre-treating step of the method of the present invention the distal end of the deformable tubular element may be provided with 4 to 15 creasing, cutting or scoring lines. Preferably the deformable tubular element may be provided with 6 to 12 creasing, cutting or scoring lines. Preferably the deformable tubular element may be provided with 8 to 10 creasing, cutting or scoring lines. The more creasing, cutting or scoring lines are provided, the better the deformable tubular element may be folded into a cylindrical form. However, with increasing number of creasing, cutting or scoring lines the complexity of the folding process increases. For typical paper material used in manufacturing the inhaler article having a diameter of about 7.2 millimeters a number of 8 to 10 creasing, cutting or scoring lines have proven to yield best results.

In general the creasing, cutting or scoring lines may be formed such as to extend parallel to the longitudinal axis of the deformable tubular element. However, these lines can also be formed to extend under any desired angle with respect to the longitudinal axis of the inhaler article. These lines may be formed to extend under an angle of between 0 to 45 degrees with respect to the longitudinal axis of the inhaler article.

After the pretreating step, the pre-treated portion of the deformable tubular element having a reduced structural stability is folded inwards by at least 90 degrees to at least partially close the distal end.

Folding the pre-treated portion may be performed in a single step. Preferably, folding the distal end of the deformable tubular element comprises a first folding step and a second folding step.

By using two folding steps a more reliable folding result may be achieved. This is mainly because by using two folding steps, folding tools with differently shaped folding heads may be used. The first folding head, which is the folding head used in the first folding step, may have an engagement surface that has a concave shape.

The second folding head, which is the folding head used in the second folding step, may have a differently shaped engagement surface. The second folding head may have an engagement surface that has a flat or a convex shape. The second folding head may have an engagement surface with a low convexity or a high convexity.

The engagement surface defines a corresponding underlying planar surface, having the same borders and being perpendicular to the longitudinal axis of the folding head.

A low convexity engagement surface is defined hereby as a surface curved, rounded or protruding outward from said underlying planar surface less than 10% of the diameter of said underlying planar surface.

A high convexity engagement surface is defined hereby as a surface curved, rounded or protruding outward from said underlying planar surface more than 10% of the diameter of said underlying planar surface.

In the first folding step the pretreated portion may be folded inwardly by an angle of less than 90 degrees. In the first folding step the pretreated portion may be folded inwardly by an angle of between 70 and 90 degrees.

In the second folding step the pretreated portion may be folded inwardly by an angle of more than 90 degrees. In the second folding step the pretreated portion may be folded inwardly by an angle of between 90 and 110 degrees.

During the pre-treatment step and during the one or more folding steps the inhaler article may be slightly rotated around its longitudinal axis with respect to the respective pre-treatment or folding head. By such rotational movement the creasing, cutting or scoring lines may be provided with a slightly helical shape. A helical shape of the creasing, cutting or scoring lines may have beneficial effects upon opening of the closed end during insertion of the inhaler article into an inhaler device.

According to a further embodiment there is provided a method for manufacturing a double length inhaler article by providing a double length mouthpiece element and a double length deformable tubular element. The double length mouthpiece element is provided in the centre of the double length deformable tubular element. Manufacturing of the double length inhaler article is largely identical as described above with the difference that both open ends of the deformable tubular element are processed at the same time. After processing the double length inhaler article is cut in the middle to obtain two identical normal length inhaler articles. Manufacturing time can be significantly reduced by processing double length inhaler articles.

The present invention is also directed to a device for manufacturing an inhaler article, comprising a body, a capsule cavity holding a capsule, a mouthpiece element and a deformable tubular element having an open end. In the pretreatment station the distal end of the deformable tubular element is pretreated to obtain a pretreated portion with reduced structural stability. In the folding station the pretreated portion is folded inwards by at least 90 degrees to at least partially close the distal end of the deformable tubular element.

The device of the present invention allows using standard foldable materials such that inhaler articles can be produced cost efficiently.

Moreover, the device allows fast and highly reproducible manufacturing of inhaler articles. The manufacturing device of the present invention can therefore be integrated in existing manufacturing lines used for production of aerosol-generating articles.

The pre-treatment station may include a processing head for creasing, cutting or scoring the distal end of the deformable tubular element.

The length of the crimping, scoring or cutting lines may be in a range from 0.5 to 5 millimeter, preferably from about 1 to 4 millimeters, and preferably from about 2.5 to 3.5 millimeters. Generally the length of these lines determines the length of the pre-treated portion with reduced structural stability.

The required length of the pre-treated portion depends on the diameter of the inhaler article.

Typical inhaler articles may have a diameter of 7.2 millimeters. For such articles the useful length of the pre-treated portion may be at least about 3 millimeters and may be at most equal to the radius (3.6 millimeters). With a pre-treated portion of such dimensions, a sufficient closure of the distal end of the deformable tubular element may be achieved.

In the pre-treating station of the present invention the distal end of the deformable tubular element may be provided with 4 to 15 creasing, cutting or scoring lines. Preferably the deformable tubular element may be provided with 6 to 12 creasing, cutting or scoring lines. Preferably the deformable tubular element may be provided with 8 to 10 creasing, cutting or scoring lines. The more creasing, cutting or scoring lines are provided, the better the deformable tubular element may be folded into a cylindrical form. However, with increasing number of creasing, cutting or scoring lines the complexity of the folding process increases. For typical paper material used in manufacturing the inhaler article having a diameter of about 7.2 millimeters a number of 8 to 10 creasing, cutting or scoring lines have proven to yield best results.

The processing head of the pre-treating station may define a generally cylindrical recess, having an inner dimension that corresponds to the outer diameter of the distal end of the deformable tubular element.

The processing head of the pre-treating station may further comprise a number of treatment blades that extend from the open side wall of the recess of the processing head towards the inner volume of the processing head. The treatment blades may extend funnel shaped towards the inner volume of the processing head. The treatment blades may be spaced equidistantly over the circumference of the recess.

The treatment blades may each have an engagement edge that contacts the distal end of the deformable tubular element during the pre-treatment step. The treatment blades may be formed such as to crease, cut or score the distal end of the deformable tubular element during the pre-treatment step.

The number of the treatment blades determines the number of the creasing, cutting or scoring lines provided to the distal end of the deformable tubular element during the pre-treatment step.

The folding station comprises at least one folding head for folding the pre-treated portion of the deformable tubular element inwards by at least 90 degrees. The folding station may comprise two folding stations, a pre-folding station and an end-folding station.

The pre-folding station may comprise a concavely shaped folding head for folding the pre-treated portion of the deformable tubular element inwards by an angle that is smaller than 90 degrees. The folding head of the pre-folding station may be designed such that the pre-treated portion of the deformable tubular element is folded inwards by an angle of between 70 and 90 degrees.

The end-folding station may comprise a flat folding head for folding the pre-treated portion of the deformable tubular element inwards by an angle of about 90 degrees. The end-folding station may also comprise a convexly shaped folding head for folding the pre-treated portion of the deformable tubular element inwards by an angle of larger than 90 degrees.

In the second folding step the pretreated portion may be folded inwardly by an angle of more than 90 degrees. In the second folding step the pretreated portion may be folded inwardly by an angle of between 90 and 110 degrees.

The pretreatment station and the folding stations of the manufacturing device may have a similar general construction. These stations may comprise a pocket for holding the tubular shaped inhaler article, in which the upstream end of the deformable tubular element is provided with a mouthpiece element and in which the distal end of the deformable tubular element is still open. Each of the processing heads of the pretreatment and the folding stations may be movably mounted opposite to and in linear alignment with the distal end of the deformable tubular element. Each of the processing heads is further configured for axial motion towards the distal end of the deformable tubular element.

For carrying out the pretreatment or the folding step, the processing head of the pretreatment or the folding station are positioned in axial alignment with the pocket holding the tubular shaped inhaler article. Once that inhaler article is correctly positioned, the processing head is moved towards the deformable distal end of the deformable tubular element. Movement of the advancement mechanism of the processing heads is controlled via a control unit. In particular, movement speed and the maximum advancement extent may be adjusted.

The advancement mechanism of each processing station is generally configured to axially move the pocket holding the inhaler article towards the respective processing head. For this purpose the processing head or the pocket or both may be axially movable. In order to reduce complexity of the processing stations, it might be advantageous that either the pocket or the processing heads are configured moveable. It might be further advantageous that only the processing heads are axially moveable. This might be particularly advantageous, if the pocket is provided with a further movable support for moving the inhaler article between the individual processing heads.

The pocket may also be provided with a movable support. The movable support may be used to position the pocket holding the tubular shaped inhaler article in each of the processing stations. The pocket may further be configured to carry the inhaler article from one processing station to the next processing station.

The pretreatment station, the pre-folding station and the end folding station may be located one after the other in processing direction, such that a linear motion of the moveable support of the pocket is sufficient for carrying the pocket with the tubular shaped article from one processing station to the next processing station.

The advancement mechanism and the moveable support may be equipped with any kind of drive mechanism. The advancement mechanism and the moveable support may be equipped with mechanical, electro-mechanical, hydraulic or pneumatic drive elements. The drive mechanism and the drive elements are connected to a control unit for setting and adjusting the appropriate movement parameters.

The pretreatment station, the pre-folding station and the end-folding station may be located one after the other in processing direction, such that a linear motion of the moveable support of the pocket is sufficient for carrying the pocket with the tubular shaped article from one processing station to the next processing station.

The pocket may also be mounted on a rotating wheel. The wheel may be configured to turn stepwise and to position the pocket holding the tubular shaped inhaler article one after the other in each of the processing stations. The wheel may be provided with a plurality of pockets such that a plurality of inhaler articles can be simultaneously carried from one processing station to the next processing station. By using a wheel with a plurality of pockets, a high speed manufacturing device may be implemented which allows for fast manufacturing of inhaler articles.

If the pockets are mounted on a rotating wheel, the pretreatment station, the pre-folding station and the end-folding station may be located one after the other in processing direction, such that the rotational motion of the rotating wheel is sufficient for carrying the pocket with the tubular shaped article from one processing station to the next processing station.

The advancement mechanism of one or more of the stations may be equipped with an end-stroke spacer. An end-stroke spacer may be used to limit the axial movement of the drive elements. This might be particularly useful when pneumatic drive elements are used for the advancement mechanism. With such end-stroke spacers the maximum extension of a pneumatic drive element may be limited. Accordingly, end-stroke spacers allow using a higher folding pressure and at the same time prevent damage to the product can be used in pneumatic drive elements while at the same time preventing damage to the product caused by overshooting movement of the drive elements.

The end-stroke spacers may be tubular cylindrical elements. In addition to limiting the axial movement of the drive elements, the end-stroke spacers may also structurally support the deformable tubular element during processing. The deformable tubular element is pressed between the end-stroke spacer and the processing head, such that the deformable tubular element folding is firmly guided during the folding processes.

Each of the processing stations may be configured to rotate the inhaler article during processing slightly around its longitudinal axis with respect to the respective processing head. By such rotational movement the creasing, cutting or scoring lines may be provided with a slightly helical shape. Advantageously, the pocket holding the inhaler article may be provided with a rotation mechanism which rotates the inhaler article during processing. In this way the rotation mechanism of the pocket can be used for rotating the inhaler article in each of the processing stations. A helical shape of the creasing, cutting or scoring lines may have beneficial effects upon opening of the closed end during insertion of the inhaler article into an inhaler device.

The processing stations can also be configured for manufacturing a double length inhaler article. For this purpose the processing stations are configured such that the double length inhaler article is held at a central portion and in either processing station processing heads are provided at either end of the double length inhaler article. Treatment of the open ends of the double length inhaler article may be as described above. An additional processing station may be provided for cutting the double length inhaler article into two normal length inhaler articles. Processing double length inhaler articles allows for increased manufacturing speed.

The present invention is also directed to the inhaler article obtainable by the manufacturing method described herein. The inhaler article comprises a deformable tubular element having a proximal and a distal end. The distal end of the deformable tubular element may be provided with 4 to 15 creasing, cutting or scoring lines. Preferably the deformable tubular element may be provided with 6 to 12 creasing, cutting or scoring lines. Preferably the deformable tubular element may be provided with 8 to 10 creasing, cutting or scoring lines.

All scientific and technical terms used herein have meanings commonly used in the art unless otherwise specified. The definitions provided herein are to facilitate understanding of certain terms used frequently herein.

As used herein, the singular forms “a”, “an”, and “the” encompass embodiments having plural referents, unless the content clearly dictates otherwise.

The term “nicotine” refers to nicotine and nicotine derivatives such as free-base nicotine, nicotine salts and the like.

The term “flavourant” or “flavour” refers to organoleptic compounds, compositions, or materials that alter and are intended to alter the taste or aroma characteristics of nicotine during consumption or inhalation thereof.

The terms “upstream” and “downstream” refer to relative positions of elements of the holder, inhaler article and inhaler systems described in relation to the direction of inhalation air flow as it is drawn through the body of the holder, inhaler article and inhaler systems.

As used herein, “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open-ended sense, and generally mean “including, but not limited to”. It will be understood that “consisting essentially of”, “consisting of”, and the like are subsumed in “comprising,” and the like.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Features described in relation to one embodiment may equally be applied to other embodiments of the invention.

The invention will be further described, by way of example only, with reference to the accompanying drawings in which:

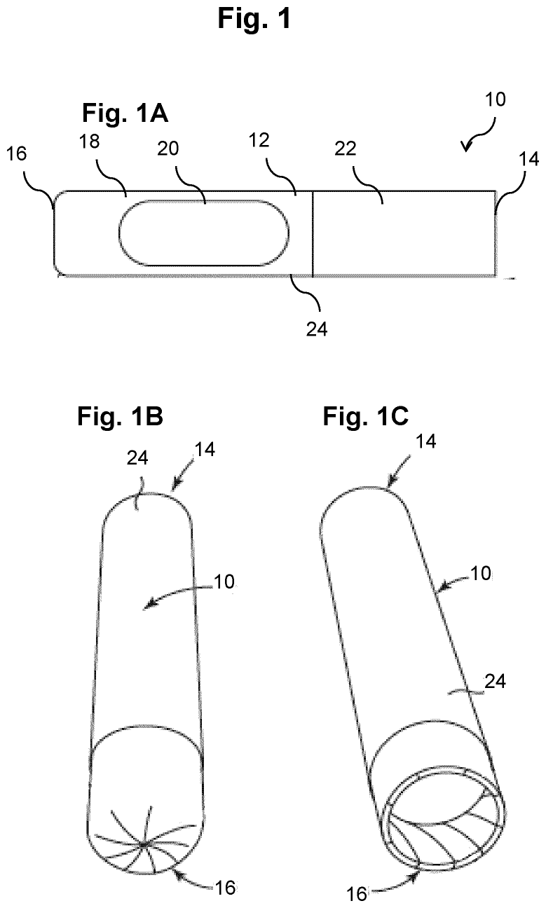

FIG. 1A is a cross-sectional schematic diagram of an illustrative inhaler article;

FIG. 1B is a front perspective view of an inhaler article with a closed distal end;

FIG. 1C is a front perspective view of an inhaler article with an open distal end;

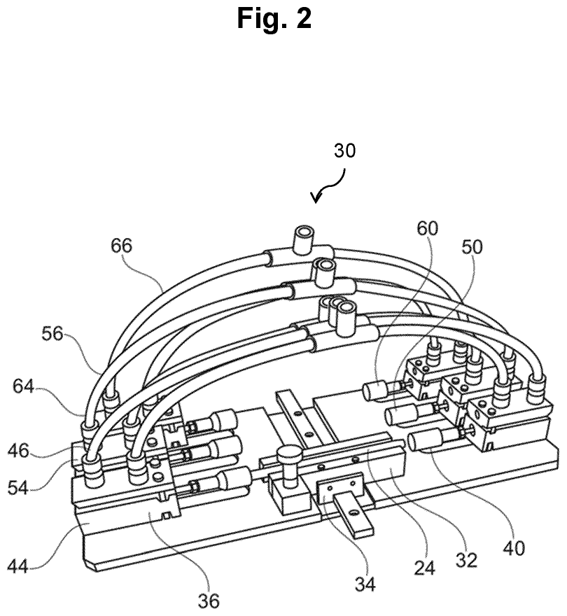

FIG. 2 is a front perspective view of a manufacturing device for an inhaler article;

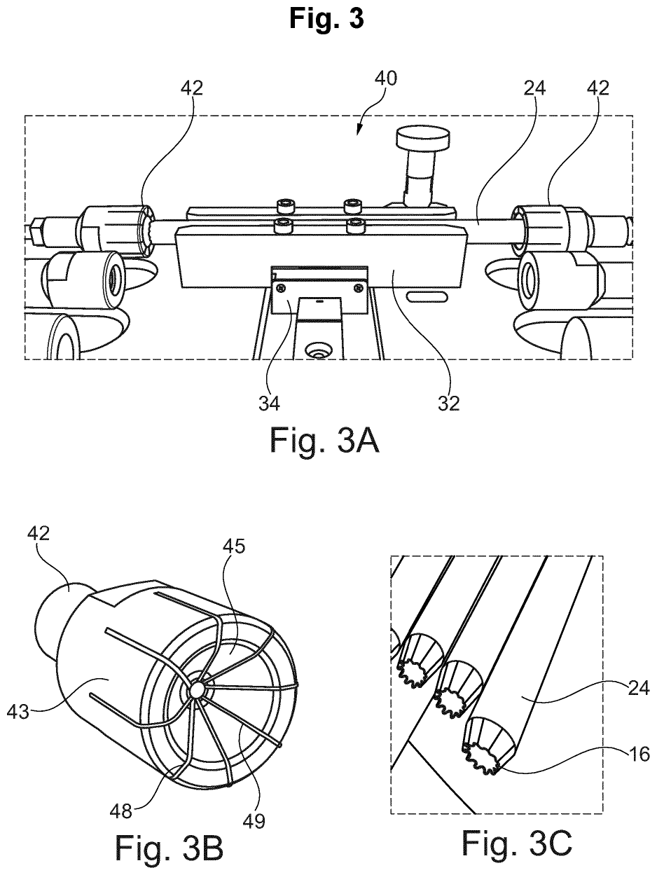

FIG. 3 shows a pretreatment station and an inhaler article after pretreatment;

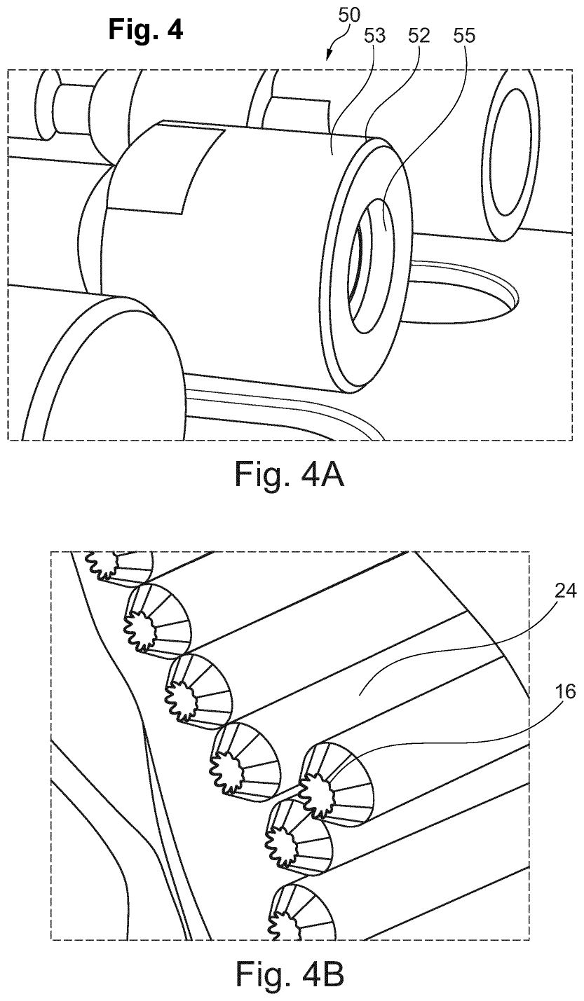

FIG. 4 shows a pre-folding station and an inhaler article after pre-folding;

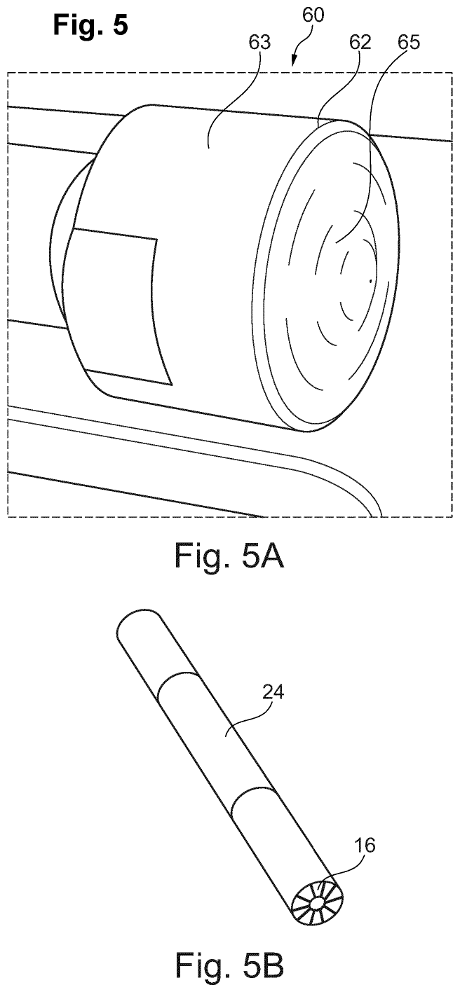

FIG. 5 shows an end-folding station and an inhaler article after end-folding;

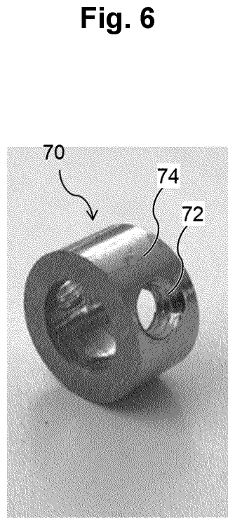

FIG. 6 is a front perspective view of an end-stroke spacer;

FIG. 1A is a cross-sectional schematic diagram of an illustrative inhaler article 10. The inhaler article 10 includes a body 12 extending along a longitudinal axis of the inhaler article 10 from a mouthpiece end 14 to a distal end 16, a capsule cavity 18 and a capsule 20 retained within the capsule cavity 18. The body 12 comprises a paper material wrapped around a mouthpiece element 22 forming a deformable tubular element 24. The deformable tubular element 24 defines the capsule cavity 18, which is bounded downstream by mouthpiece element 22 and which is bounded upstream by the at least partially closed distal end 16 of the deformable tubular element 24.

In the embodiment of FIG. 1 the deformable tubular element 24 is formed of paper having a thickness of about 125 micrometers and a basis weight of about 100 grams per square meter. The illustrated inhaler article 10 has a mouthpiece element length of about 20 mm and the deformable tubular element 24 has a length of about 45 mm with an outer uniform diameter of about 7.2 mm.

FIG. 1B is a front perspective view of the illustrative inhaler article 10 wherein the distal end 16 of the deformable tubular element 24 is closed. The deformable tubular element 24 is folded back onto itself forming overlapping pie shaped sections closing the distal end 16 of the capsule cavity 18.

FIG. 1C is a front perspective view of the illustrative inhaler article with a deformable tubular element 24 wherein the distal end 16 is opened. The folded sections of the distal end 16 of the deformable tubular element 24 may be opened to expose the capsule cavity 18. For opening the distal end 16 the deformable tubular element 24 may be inserted into an appropriate holder, not described herein. After the folded sections of the distal end 16 of the deformable element 24 are opened, an aperture for receiving swirling or rotating inhalation airflow is formed.

FIG. 2 shows a device 30 for automatically processing an inhaler article to form a closed distal end 16. The device as depicted in FIG. 2 is configured for use of a double length inhaler article having a double length mouthpiece element 22 and a double length deformable tubular element 24.

The device 30 comprises a pre-treatment station 40, a pre-folding station 50 and an end-folding station 60. The double length deformable tubular element 24 that is already combined with the mouthpiece element is provided to pocket 32 that is movable in processing direction from the pre-treatment station 40 to the pre-folding station 50 and further to the end-folding station 60. In this embodiment pocket 32 is mounted on moveable support 34 and is manually movable.

Each of the pre-treatment station 40, pre-folding station 50 and end-folding station 60 comprise a processing head 42, 52, 62 at either side of the pocket. Each of the processing heads is equipped with an advancement mechanism 36 that comprises pneumatic drive elements 44, 54, 64. The pneumatic drive elements are provided with pressurized air via air ducts 46, 56, 66. The advancement mechanism 36 is controlled via a central control unit (not shown).

The individual processing stations are discussed in more detail below with respect to FIGS. 3 to 7.

In FIG. 3 an embodiment of a pre-treatment station 40 is depicted. In the centre of FIG. 3 pocket 32 holding of a double length inhaler article is shown. Pocket 32 is mounted on a movable support 34 via which pocket 32 can be positioned at the various processing stations. At either side of the pocket 32 a crimping head 42 is provided. Each crimping head 42 is movable by an advancement mechanism 36 (not visible in FIG. 3), comprising pneumatic drive elements 44.

Crimping head 42 is shown in more detail in FIG. 3B. The crimping head 42 defines a generally cylindrical body 43 with an open end 45 for insertion of the distal end 16 of the deformable tubular element 24 of the inhaler article 10. The crimping head 42 comprises eight crimping blades 48 that are mounted to the body 43 of the crimping head 42. The crimping blades 48 extend from the rim of the open end 45 into the interior volume of the crimping head 42. The treatment blades 48 are spaced equidistantly over the circumference of the rim of the open end 45 and extend funnel-shaped towards the inner volume of the crimping head 42.

Each of the crimping blades 48 has an engagement edge 49 that contacts the distal end of the deformable tubular element 24 during crimping. During the crimping process the crimping head is moved axially towards the pocket 32 holding the inhaler article 10. The crimping blades 48 contact the distal end 16 of the deformable tubular element 24. After the crimping process the distal end 16 of the deformable tubular element 24 looks as depicted in FIG. 3C. The ends of the deformable tubular element 24 are slightly bent inward and are provided with crimping lines having a length of about 3.5 millimeters.

In FIGS. 4A and 5A processing heads of the pre-folding station and the end-folding station are depicted. The processing head 52 of the pre-folding station 50 has also a generally cylindrical body 53 having a concavely shaped engagement surface 55.

During the pre-folding process the pre-folding head 52 is moved axially towards the pocket 32 holding the inhaler article 10. The concavely shaped engagement surface 55 contacts the pre-treated distal end 16 of the deformable tubular element 24. After the pre-folding process the distal end 16 of the deformable tubular element 24 looks as depicted in FIG. 4B. The ends of the deformable tubular element 24 are now bent inward along the crimping lines. The folding angle is well below 90 degrees.

After the pre-folding station the inhaler article is carried to the end-folding station 60. The processing head 62 of the end-folding station 60 has a generally cylindrical body 63 having a convexly shaped engagement surface 65.

During the end-folding process the end-folding head 62 is moved axially towards the pocket 32 holding the inhaler article 10. The convexly shaped engagement surface 64 contacts the pre-folded distal end 16 of the deformable tubular element 24. After the end-folding process the distal end 16 of the deformable tubular element 24 looks as depicted in FIG. 5B. The ends of the deformable tubular element 24 are now bent inward at a folding angle of about 90 degrees. In the centre of the folded distal end 16 a residual opening with a diameter of between 0.5 and 1 millimetre is obtained.

In order to structurally support the distal end 16 of the deformable tubular element 24 during pre-folding and end-folding the folding heads 52, 62 are provided with ring shaped end-stroke spacers 70 as depicted in FIG. 6. The end-stroke spacers 70 are mounted to the folding heads 52, 62 via screws that are inserted into threading 72 provided in the side walls 74 of the end-stroke spacers 70. The end-stroke spacers 70 are provided in vicinity to the crimped area and guide the folding movement of the distal end 16 of the deformable tubular element 24. The end-stroke spacers 70 can be provided around the crimped end or in the advancement mechanism 36, so as to limit the axial movement of the advancement mechanism.

After folding both its ends, the double length inhaler article 24 is cut in the middle, to obtain two inhaler articles with closed distal ends 16. Cutting can be performed with conventional cutting devices.

1. Method for manufacturing an inhaler article, the inhaler article comprising a body, a capsule cavity holding a capsule, a mouthpiece element and a deformable tubular element having an open distal end, the method comprising:
pre-treating the distal end of the deformable tubular element to obtain a pre-treated portion with reduced structural stability, and
folding the pre-treated portion inwards by at least 90 degrees to at least partially close the distal end, wherein folding the distal end of the deformable tubular element comprises a pre-folding step and an end-folding step and, wherein the pre-folding step comprises folding the pre-treated portion of the deformable tubular element inwards by an angle that is smaller than 90 degree by means of concavely shaped folding head.

2. Method according to claim 1, wherein pretreating the distal end of the deformable tubular element comprises cutting, scoring or crimping the edge of the distal end of the deformable tubular element.

3. Method according to claim 1, wherein pretreating the distal end of the deformable tubular element comprises providing 8 to 10 cutting, scoring or crimping lines to the edge of the distal end of the deformable tubular element.

4. Method according to claim 1, wherein the end-folding step comprises folding the pre-folded portion of the deformable tubular element inwards by an angle of about 90 degrees by means of a flat folding head.

5. Method according to claim 1, wherein the end-folding step comprises folding the pre-folded portion of the deformable tubular element inwards by an angle of larger than 90 degrees by means of a convexly shaped folding head.

6. Device for manufacturing an inhaler article, the inhaler article comprising a body, a capsule cavity holding a capsule, a mouthpiece element and a deformable tubular element having an open distal end, the device comprising:
a pre-treatment station in which the distal end of the deformable tubular element is pre-treated to obtain a pre-treated portion with reduced structural stability, and
a folding station in which the pre-treated portion is folded inwards by at least 90 degrees to at least partially close the distal end of the deformable tubular element, wherein the folding station comprises a pre-folding station comprising a concavely shaped folding head for folding the pre-treated portion of the deformable tubular element inwards by an angle that is smaller than 90 degrees.

7. Device according to claim 6, wherein the pre-treatment station includes a pre-treatment head for cutting, scoring or crimping the distal end of the deformable tubular element.

8. Device according to claim 7, wherein the pre-treatment head comprises edges for providing 8 or 10 cutting, scoring or crimping lines to the distal end of the deformable tubular element.

9. Device according to claim 6, wherein the folding station comprises at least one folding head for folding the pre-treated portion of the deformable tubular element inwards by at least 90 degrees.

10. Device according to claim 6, wherein the folding station comprises an end-folding station comprising a flat folding head for folding the pre-treated portion of the deformable tubular element inwards by an angle of about 90 degrees.

11. Device according to claim 6, wherein the folding station comprises an end-folding station with a convexly shaped folding head for folding the pre-treated portion of the deformable tubular element inwards by an angle of larger than 90 degrees.

12. Device according to claim 6, wherein one or more of the pre-treatment station and the folding station comprise an advancement mechanism configured to move the respective processing head towards the deformable tubular element.

13. Device according to claim 6, wherein one or more of the pre-treatment station and the folding station comprise an end-stroke spacer to limit the axial movement of the drive elements of the advancement mechanism.

14. Device according to claim 13, wherein the one or more end-stroke spacers are tubular cylindrical elements that structurally support the deformable tubular element during processing.
